(19) United States
(12) Patent Application Publication
(10) Pub. No.: US 2018/0221646 A1
(43) Pub. Date: Aug. 9, 2018

(54) Wireless Electrodes

(71) Applicant: Jennifer Silverton, Arlington, TX (US)

(72) Inventor: Jennifer Silverton, Arlington, TX (US)

(21) Appl. No.: 15/891,128

(22) Filed: Feb. 7, 2018

Related U.S. Application Data
(60) Provisional application No. 62/456,720, filed on Feb. 9, 2017.

(51) Int. Cl. A61N 1/04; A61B 5/0404; A61B 5/00

(52) U.S. Cl. CPC A61N 1/048; A61B 5/0205; A61B 5/0006; A61B 5/0404

(57) ABSTRACT

The present invention relates to wireless electrodes capable of being used with electrocardiogram readers. The wireless electrodes eliminate the need for wiring between electrodes and the electrocardiogram reader. In one embodiment of the present invention, the wireless electrodes are nested in a base station. The base station pairs all the wireless electrodes via at least one of a Bluetooth connection, an Infrared Connection, and a Wireless Local Area Network Connection. Data received from the wireless electrodes is synthesized and exported to the base station. The base station transmits the data into the electrocardiogram reader. In one example, the base station serves as a charging station for the wireless electrodes. The base station comprises a plurality of nesting ports. The wireless electrodes fit into the plurality of nesting ports. Further, the plurality of nesting ports comprises charging pads. The charging pads charge the wireless electrodes.

### BACKGROUND OF THE INVENTION

### A. Technical Field

The present invention generally relates to electrocardiogram readers. The invention is specifically related to wireless electrodes capable of being used with the electrocardiogram reader.

### B. Description of Related Art

The latter half of twentieth century has witnessed the advent of a wide variety of electronic devices into the daily lives of common people. Electronic devices are used by almost every person on earth, in one form or another. In several professions, professionals rely on an assortment of electronic devices to carry out daily duties and to render services to customers. The medical profession is no exception and is ever more dependent on highly sophisticated electronic devices. Examples of electronic devices used in the medical profession include, but are not limited to electronic thermometers, electrocardiogram readers, and ultrasound scanners.

Even though a wide gamut of medical equipments today use wireless technology, devices such as electrocardiogram readers are known to be profoundly wire dependent. Electrocardiogram readers require a vast assemblage of complex wiring to be connected to a patient's body via electrodes. The complex wiring of electrocardiogram reader can intimidate the patient and create unnecessary stress and anxiety in the patient's mind. Even though operation of the electrocardiogram readers by itself is not painful for the patient, the stress and anxiety induced as a result of the complex wiring in the electrocardiogram readers can prove contrary to general well-being of the patient. Further, connecting the complex wiring properly on to the patient, may prove to be a daunting task for health care professionals as well. Moreover, the complex wiring and electrodes consume storage space. In one example, an electrocardiogram reader requires twelve electrodes to be attached on a patient's body. The twelve electrodes are attached to the patient for recording electrical activity of the patient's heart. Each of the twelve electrodes is connected to the electrocardiogram reader via a different wire. Problems associated with the complex wirings between the electrodes and the electrocardiogram readers may be alleviated by using wireless electrodes.

Therefore, there is a need for wireless electrodes capable of being used in medical devices.

### SUMMARY OF THE INVENTION

The present invention discloses a wireless electrode arrangement for physiological monitoring of a patient. The wireless electrode arrangement capable of being used with electrocardiogram readers. The wireless electrode arrangement eliminates the need for wiring between electrodes and an electrocardiogram reader.

In an embodiment, the wireless electrode arrangement comprises a plurality of wireless electrode attachable to the patient, which is configured to acquire electrical signal from the patient. Then the acquired electrical signal is synthesized. The plurality of wireless electrode is further configured to wirelessly transmit the synthesized electrical signal to a base station. The base station is configured to receive the synthesized electrical signals from each wireless electrode, and wirelessly transmit the electrical signals to a medical device such as electrocardiogram reader.

In one embodiment of the present invention, each wireless electrode is nested in a base station. The base station pairs all the wireless electrodes via at least one of a Bluetooth connection, an Infrared connection, and a Wireless Local Area Network connection. Furthermore, each wireless electrode have internal batteries. In one example, the base station serves as a charging station for the plurality of wireless electrodes. The base station comprises a plurality of nesting ports to nest each wireless electrode. Further, the plurality of nesting ports comprises charging pads. The charging pads is configured to charge the wireless electrodes.

Furthermore, each wireless electrode comprises one or more LED lights. The one or more lights flash or emit light when the plurality of wireless electrodes are charged in the plurality of nesting ports. Furthermore, the base station has an internal power source. The internal power source allows the base station to be used in situations wherein external power is unavailable. The base station further comprises at least one LED configured to emit light on charging the plurality of wireless electrode. Each wireless electrode further comprises at least one disposable adhesive probe. The disposable adhesive probes are attached to a spring clip disposed on each wireless electrode.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 is a block diagram of an environment implemented in accordance with various embodiments of the invention.

FIG. 2 shows an exploded view of an adhesive probe of the wireless electrode, incorporating aspects of the present invention.

FIG. 3 shows the wireless electrode, incorporating the aspects of the present invention.

FIG. 4 shows a base station nesting a plurality of wireless electrodes, incorporating the aspects of the present invention.

FIG. 5 shows the base station receiving the wireless electrode onto the nesting port, incorporating the aspects of the present invention.

FIG. 6 shows the plurality of wireless electrodes attached to a patient's body, incorporating the aspects of the present invention.

### DETAILED DESCRIPTION OF EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive.

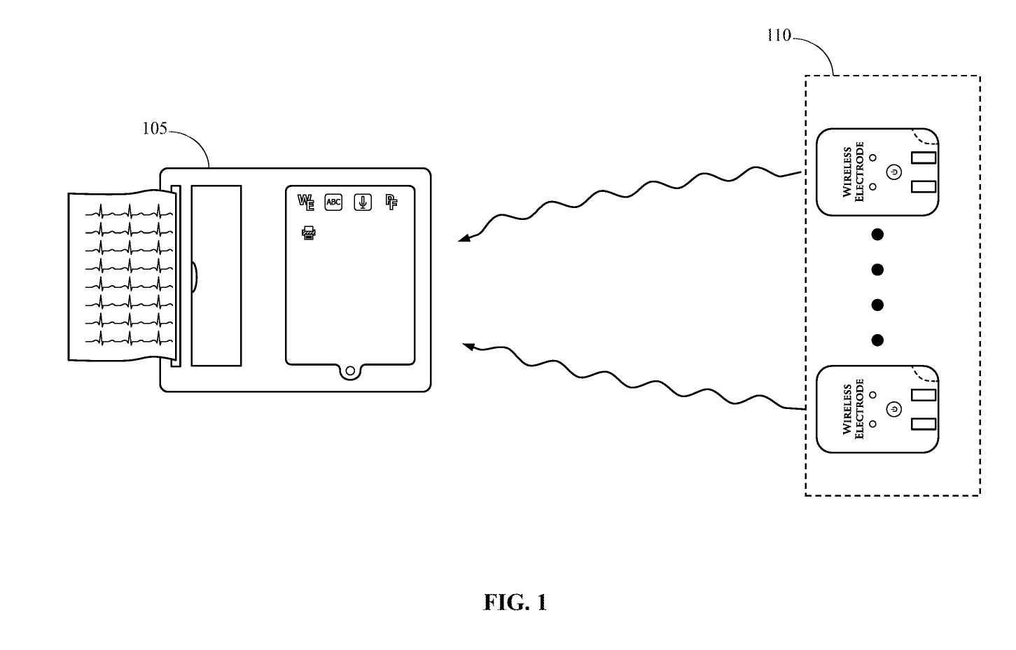

According to FIG. 1, the present invention discloses a wireless electrode arrangement. The wireless electrode arrangement comprises a plurality of wireless electrodes 110 wirelessly connected to an electrocardiogram reader 105. In one embodiment of the present invention, the plurality of wireless electrodes 110 are wirelessly connected to the electrocardiogram reader 105 via a base station (not shown). The plurality of wireless electrodes 110 are connected to the electrocardiogram reader 105 and the base station via at least one of a Bluetooth network, an infrared connection and a Wireless Local Area Network.

The plurality of wireless electrode 110 attachable to a patient is configured to acquire electrical signal from the patient. Then the acquired electrical signal is synthesized. The plurality of wireless electrode 110 is further configured to wirelessly transmit the synthesized electrical signal to a base station. The base station is configured to receive the synthesized electrical signals from each wireless electrode 110, and wirelessly transmit the electrical signals to a medical device such as electrocardiogram reader 105.

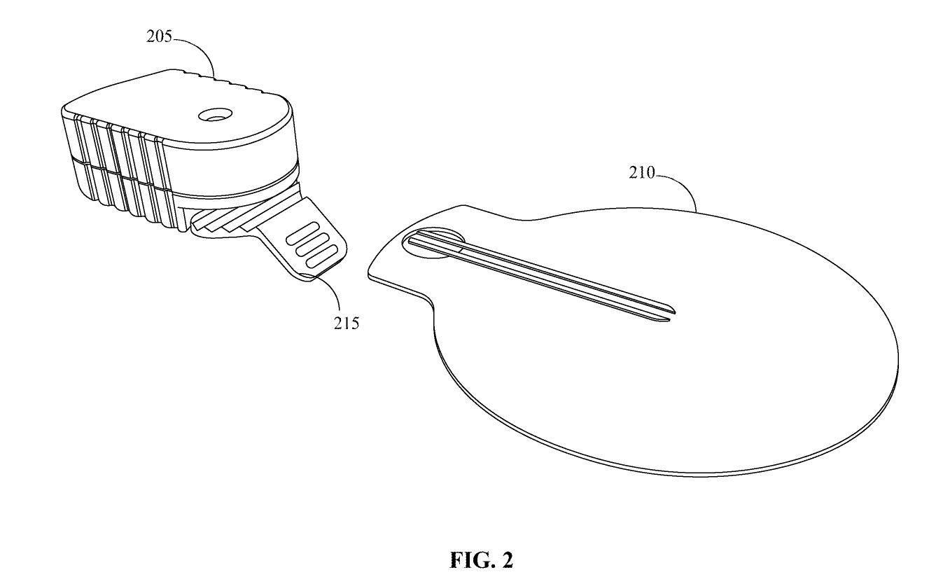

Referring to FIG. 2, a wireless electrode 205 and an adhesive probe 210 are attachable to each other via a spring clip 215 disposed on the wireless electrode 205. The wireless electrodes 205 further comprise at least one disposable adhesive probe 210. The disposable adhesive probes 210 are attached to a spring clip 215 disposed on the wireless electrodes 205.

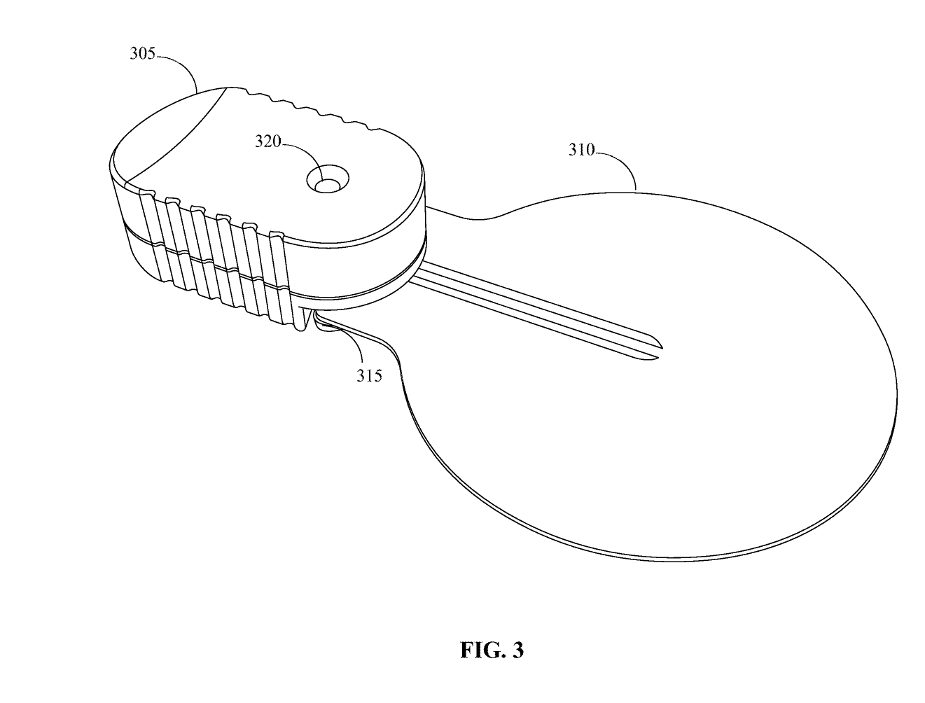

Referring to FIG. 3, a wireless electrode 305 is attached to an adhesive probe 310 via a spring clip 315. The wireless electrode 305 further comprises an LED light 320. The wireless electrode 305 has an internal battery (not shown). The internal battery is charged in a base station (not shown). The LED light 320 flashes when the wireless electrode 305 is being charged in the base station. The wireless electrode 305 further comprises a Bluetooth transmitter (not shown) to transmit data to the base station. Moreover, the wireless electrode 305 comprises a voltmeter to detect electrocardiographic voltages. In one example, the wireless electrode 305 is fixed to the skin of a patient on top of a layer of electrocardiogram gel.

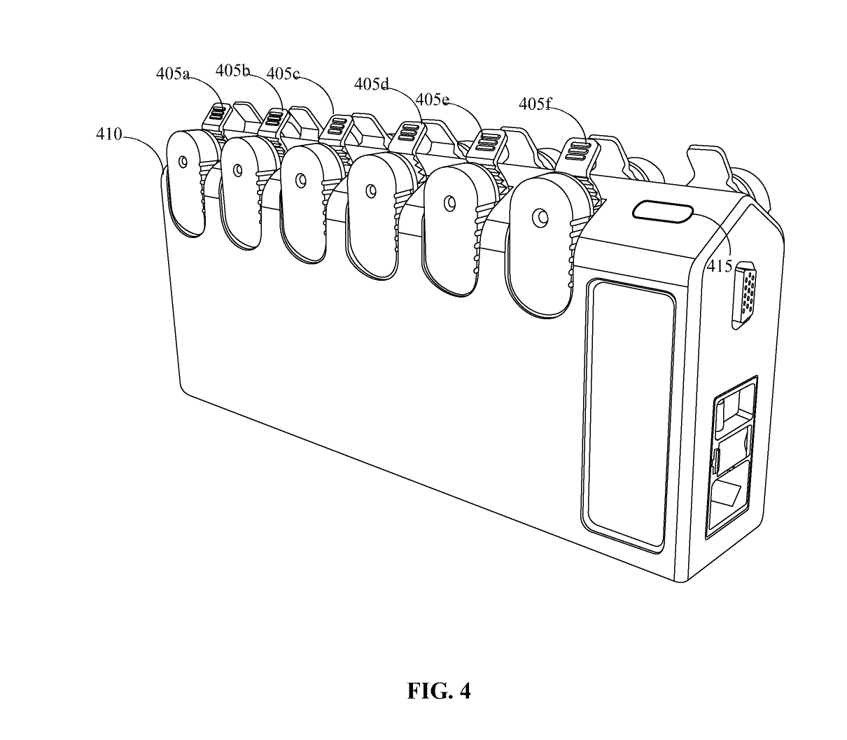

Referring to FIG. 4, a base station 410 is capable of nesting a plurality of wireless electrodes (405a, 405b, 405c, 405d, 405e, and 405f) in a plurality of nesting ports (not shown). The plurality of nesting ports has a plurality of charging pads (not shown). The plurality of wireless electrodes (405a, 405b, 405c, 405d, 405e, and 405f) charges inside the plurality of nesting ports. The base station 410 further comprises an LED light 415. The LED light 415 glows red when the plurality of wireless electrodes (405a, 405b, 405c, 405d, 405e, and 405f) need charging. The LED light 415 glows green when the plurality of wireless electrodes (405a, 405b, 405c, 405d, 405e, and 405f) is fully charged.

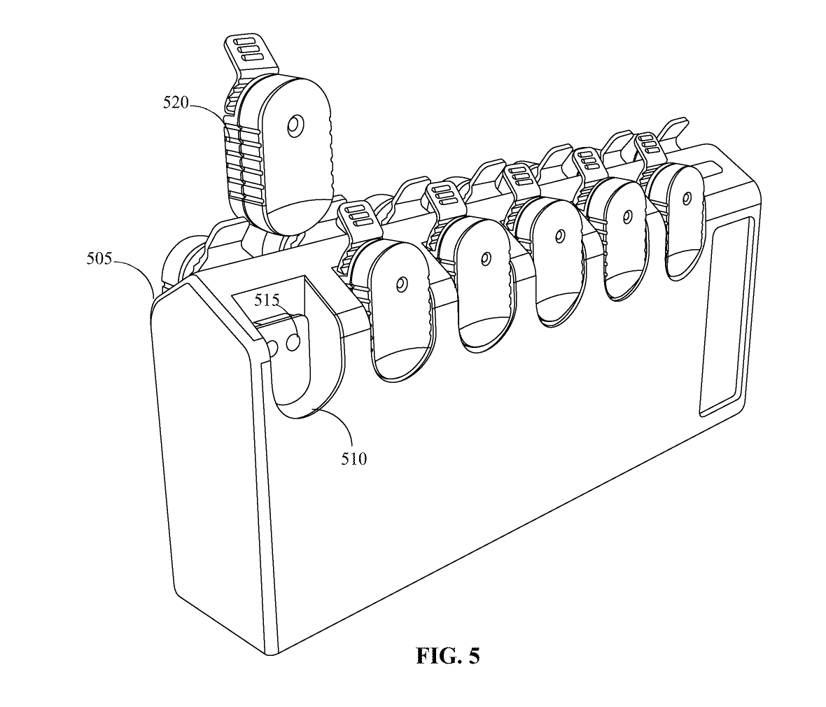

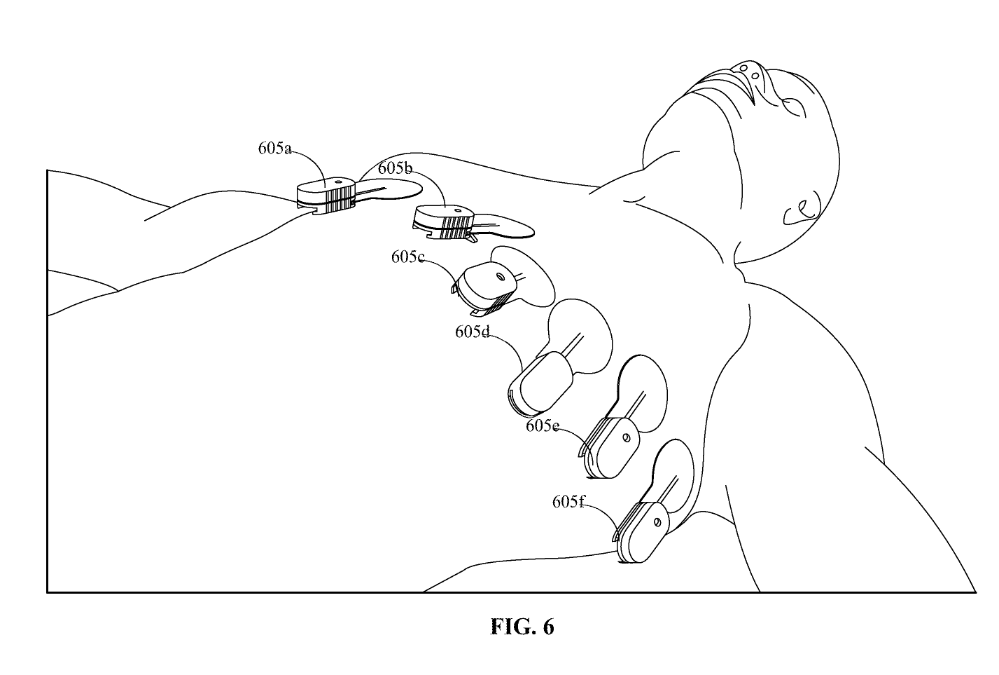

Referring to FIG. 5, a base station 505 has at least one nesting port 510. Further, a wireless electrode 520 has at least one charging pad or charging surface (not shown). Similarly, the nesting port 510 has charging pads 515. Referring to FIG. 6, a plurality of wireless electrodes (605a, 605b, 605c, 605d, 605e, and 605f) are attached on to a patient's body.

Advantageously, present invention presents an innovative method of eliminating complex wiring in electrocardiograms readers. Further, the present invention reduces patient anxiety by eliminating festoons of wiring around the patient's body. Furthermore, internal batteries in a base station and the wireless electrodes enable operation in areas lacking wherein external power sources are unavailable. Furthermore, lack of the complex wiring ensures portability to the electrocardiogram reader. Furthermore, the electrocardiogram with the wireless electrodes is easy to use and set up. Further, the wireless electrodes have a simple and straightforward design.

Preferred embodiments of this invention are described herein, including the best mode known to the inventors for carrying out the invention. It should be understood that the illustrated embodiments are exemplary only and should not be taken as limiting the scope of the invention.

The foregoing description comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein.

What is claimed is:

1. A wireless electrode arrangement for physiological monitoring of a patient, comprising:
a plurality of wireless electrode attachable to the patient configured to:
acquire and synthesize electrical signals from the patient, and
wirelessly transmit the synthesized electrical signal to a base station, wherein the base station is configured to:
receive the synthesized electrical signals from each wireless electrode, and
wirelessly transmit the received signals to a medical device.

2. The wireless electrode arrangement according to claim 1, wherein the medical device is an electrocardiogram reader.

3. The wireless electrode arrangement according to claim 1, wherein the base station further comprises a plurality of nesting port.

4. The wireless electrode arrangement according to claim 3, wherein each nesting port is configured to receive each wireless electrode therein.

5. The wireless electrode arrangement according to claim 1, wherein each wireless electrode further comprises a Bluetooth transmitter to transmit the synthesized electrical signals to the base station.

6. The wireless electrode arrangement according to claim 1, wherein the base station communicates with the plurality of wireless electrodes via at one of a Bluetooth connection, an Infrared connection, and a Wireless Local Area Network connection.

7. The wireless electrode arrangement according to claim 1, wherein each wireless electrode is attached to at least one disposable adhesive probe via a spring clip.

8. The wireless electrode arrangement according to claim 1, wherein each wireless electrode further comprises an internal battery and a charging surface.

9. The wireless electrode arrangement according to claim 1, wherein the base station further comprises an internal power source and a charging pad to charge the internal battery.

10. The wireless electrode arrangement according to claim 1, wherein the base station further comprises at least one LED configured to emit light on charging the plurality of wireless electrode.

11. The wireless electrode arrangement according to claim 1, wherein each wireless electrode further comprises at least one LED.

12. The wireless electrode arrangement according to claim 11, wherein the at least one LED is configured to emit light depending on the charge status of the internal battery.

13. The wireless electrode arrangement according to claim 1, wherein each wireless electrode comprises a voltmeter to detect electrocardiographic voltages.
